(19) United States
(12) Patent Application Publication
(10) Pub. No.: US 2026/0158206 A1
(43) Pub. Date: Jun. 11, 2026

(54) DETACHABLE SPRAY AND SUCTION DEVICE FOR NASAL

(71) Applicant: Shenzhen Lute Jiacheng Supply Chain Management Co., Ltd., Shenzhen (CN)

(72) Inventors: Siyu LI, Shenzhen (CN); Qiao YAO, Shenzhen (CN); Liu LIU, Shenzhen (CN)

(73) Assignee: Shenzhen Lute Jiacheng Supply Chain Management Co., Ltd., Shenzhen (CN)

(21) Appl. No.: 19/030,971

(22) Filed: Jan. 17, 2025

(30) Foreign Application Priority Data
Dec. 6, 2024 (CN) 202423021937.5

(51) Int. Cl. A61M 1/00; A61B 17/24; A61M 3/02; A61M 11/00

(52) U.S. Cl. CPC A61M 1/772; A61B 17/24; A61M 3/0258; A61M 3/0287; A61M 11/00; A61B 2017/246; A61M 2205/0216; A61M 2205/07; A61M 2210/0618

(57) ABSTRACT

Disclosed is a detachable spray and suction device for the nasal, which includes a suction spray assembly and a host machine. The suction spray assembly includes a housing, a suction portion, and a spray portion. The housing is provided with an installation cavity, and the suction portion is connected to the spray portion; the suction portion is provided with a suction end exposed in the installation cavity, and the spray portion is provided with a spray end exposed in the installation cavity. The host machine includes an air pump and a connection air pipe. The connection air pipe is connected with the air pump and the suction spray assembly, and the air pump is provided with an air suction port and an air discharge port; the connection air pipe is connected with the air suction port and the suction portion.

### CROSS-REFERENCE TO RELATED APPLICATIONS

The present application claims priority to Chinese Patent Application No. 202423021937.5, filed on Dec. 6, 2024, the entire disclosure of which is incorporated herein by reference.

### TECHNICAL FIELD

The present application relates to the technical field of spray and suction device for the nasals, and in particular to a detachable spray and suction device for the nasal.

### BACKGROUND

A spray and suction device for a nasal is an instrument that focuses on providing cleaning or medical use. The spray and suction device for the nasal includes main structures such as a spray portion and a suction portion. In terms of home care, it is necessary to provide simple and rapid nasal or oral care, including nasal medication and suction of the user's nasal mucus or phlegm through the spray portion. In addition, the spray portion can further soften the solidified lumps in the nasal cavity, making it more convenient to use the nasal suction portion.

The existing spray and suction device for the nasal is designed as an integrated structure. After using the spray and suction device for the nasal to clean the nasal cavity or oral cavity, the user needs to clean the inside of the spray and suction device for the nasal. When cleaning the integrated spray and suction device for the nasal, the structure to be cleaned is integrated with non-waterproof structures such as air pumps and batteries, resulting in incomplete cleaning and disinfection, which in turn causes unnecessary hygiene problems and affects user use.

### SUMMARY

The main purpose of the present application is to provide a spray and suction device for a nasal, aiming to solve the problem that when the integrated spray and suction device for the nasal is cleaned, the structure to be cleaned is integrated with the non-waterproof structure such as the air pump and the battery, resulting in incomplete cleaning and disinfection, which in turn causes unnecessary hygiene problems and affects the user's use.

To achieve the above purpose, the present application provides a detachable spray and suction device for a nasal including a suction spray assembly and a host machine. The suction spray assembly includes a housing, a suction portion, and a spray portion. The housing is provided with an installation cavity, and the suction portion is connected to the spray portion; the suction portion is provided with a suction end exposed in the installation cavity, and the spray portion is provided with a spray end exposed in the installation cavity. The host machine includes an air pump and a connection air pipe. The connection air pipe is connected with the air pump and the suction spray assembly, and the air pump is provided with an air suction port and an air discharge port; the connection air pipe is connected with the air suction port and the suction portion, and the air discharge port is configured to communicate the air pump with outside world.

In an embodiment, the suction portion includes a nasal suction chamber and a chamber cover; the chamber cover is connected to the nasal suction chamber, and the nasal suction chamber is detachably provided in the installation cavity; the chamber cover and the nasal suction chamber are enclosed to form a buffer cavity; the chamber cover is provided with a nasal suction port, and the nasal suction port is configured to communicate the buffer cavity with the outside world.

In an embodiment, the housing and the nasal suction chamber are enclosed to form an air suction channel, and the connection air pipe is configured to communicate the air suction channel with the air suction port; a side wall of the nasal suction chamber is provided with an air suction port, and the air suction port is configured to communicate the buffer cavity with the air suction channel.

In an embodiment, the suction portion further includes a nasal suction tip, and the nasal suction tip is connected to the chamber cover; the nasal suction tip is provided with a nasal suction channel, and the nasal suction channel is configured to communicate the buffer cavity with the outside world.

In an embodiment, a liquid storage tank is provided in the installation cavity; a spray opening is provided at a side wall of the liquid storage tank, and the spray portion includes an atomization assembly; the atomization assembly is connected to the housing, and the atomization assembly is provided at the spray opening.

In an embodiment, the spray portion further includes a spray power structure; the spray power structure is detachably connected to the housing, and the spray power structure is electrically connected to the atomization assembly.

In an embodiment, the spray portion further includes an atomization hood; the atomization hood is detachably connected to the housing, and the atomization hood is provided at the spray opening.

In an embodiment, the host machine further includes an air channel adapter, and the air channel adapter is provided with an air suction cavity and an air discharge cavity; the air suction cavity is configured to communicate the connection air pipe with the air pump, and the air discharge cavity is configured to communicate the air pump with the outside world.

In an embodiment, a cross-sectional area of the air suction cavity along an air flow direction of the air pump is larger than a cross-sectional area of the connection air pipe, and a cross-sectional area of the discharge cavity along the air flow direction of the air pump is larger than a cross-sectional area of the discharge port.

In an embodiment, the air path adapter is made of an ethylene-vinyl acetate copolymer (EVA) material or a silicone material.

The present application provides a detachable spray and suction device for a nasal. By separating the suction spray assembly from the host machine, the detachable spray and suction device for the nasal can be used conveniently, and cleaning can be performed more conveniently and thoroughly. The suction spray assembly includes a housing, a suction portion and a spray portion. The suction portion and the spray portion are respectively provided at the two ends of the housing, so that the user can spray and suck the nose according to the needs. The host machine includes an air pump and a connection air pipe. The air pump is connected to the suction portion through the connection air pipe, to realize the suction function. After use, the user can easily disassemble the suction spray assembly for cleaning without worrying about the damage of non-waterproof components, such as the air pump and the battery, thereby achieving thorough cleaning and disinfection. The detachable design makes it easier to clean the spray and suction device for the nasal, avoiding the problem of incomplete cleaning due to the complex structure in the integrated structure. Since the non-waterproof components, such as the air pump and the battery, are separated from the suction spray assembly to be cleaned, the user can fully clean and disinfect the suction spray assembly, effectively preventing bacterial growth, reducing hygiene problems, and improving the hygiene of the product and the user experience. In addition, the detachable design may further reduce maintenance costs since each part can be replaced separately without replacing the entire device.

### BRIEF DESCRIPTION OF THE DRAWINGS

To more clearly illustrate technical solutions in the embodiments of the present application or the related art, the following will briefly introduce the drawings that need to be used in the description of the embodiments or the related art. Obviously, the drawings in the following description are only some embodiments of the present application. For those skilled in the art, without creative effort, other drawings can be obtained according to the structures shown in these drawings.

FIG. 1 is a schematic structural diagram of a detachable spray and suction device for the nasal according to an embodiment of the present application.

FIG. 2 is a schematic structural diagram of a suction spray assembly according to an embodiment of the present application.

FIG. 3 is a cross-sectional view at position A-A in FIG. 2.

FIG. 4 is a schematic structural diagram of the suction spray assembly according to another embodiment of the present application.

FIG. 5 is a schematic structural diagram of an air pump and an air channel adapter according to an embodiment of the present application.

FIG. 6 is a cross-sectional schematic structural diagram of the air channel adapter according to an embodiment of the present application.

The realization of the objective, functional characteristics, and advantages of the present application are further described with reference to the accompanying drawings.

### DETAILED DESCRIPTION OF THE EMBODIMENTS

The technical solutions of the embodiments of the present application will be described in detail below with reference to the accompanying drawings. It is obvious that the embodiments described are only some rather than all of the embodiments of the present application. All other embodiments obtained by those skilled in the art based on the embodiments of the present application without creative efforts shall fall within the claimed scope of the present application.

It should be noted that all the directional indications (such as up, down, left, right, front, rear...) in the embodiments of the present application are only used to explain the relative positional relationship, movement, or the like of the components in a certain posture. If the specific posture changes, the directional indication will change accordingly.

Besides, the descriptions associated with, e.g., “first” and “second,” in the present application are merely for descriptive purposes, and cannot be understood as indicating or suggesting relative importance or impliedly indicating the number of the indicated technical feature. Therefore, the feature associated with “first” or “second” can expressly or impliedly include at least one such feature. Further, if “and/or” appears throughout the text, it includes three parallel schemes. Taking “A and/or B” as an example, it includes the scheme A, or the scheme B, or the scheme that the scheme A and the scheme B satisfy at the same time. In addition, the technical solutions of the various embodiments can be combined with each other, but the combinations must be based on the realization of those skilled in the art. When the combination of technical solutions is contradictory or cannot be achieved, it should be considered that such a combination of technical solutions does not exist, nor does it fall within the scope of the present application.

The present application proposes a detachable spray and suction device for a nasal 100.

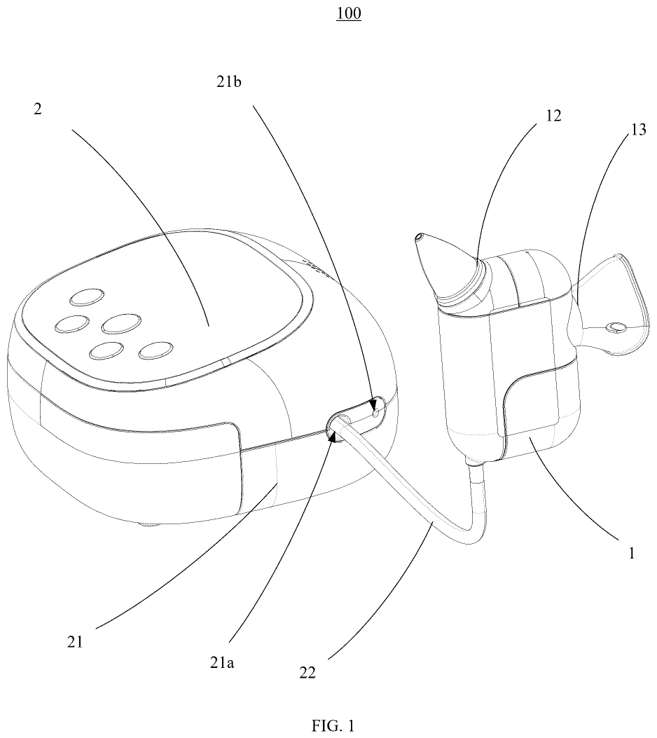

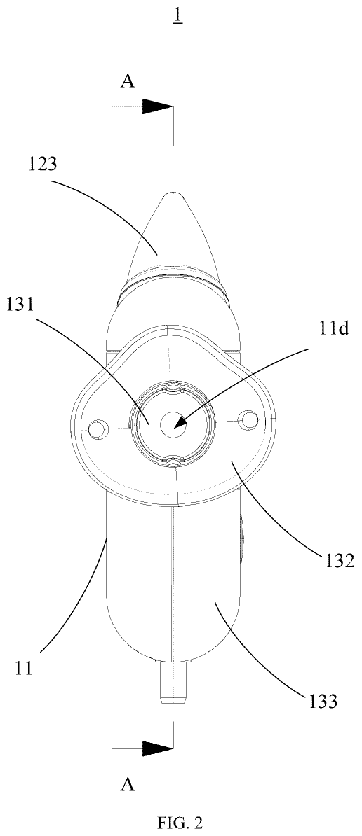

As shown in FIG. 1 to FIG. 3, in an embodiment of the present application, the detachable spray and suction device for the nasal 100 includes a suction spray assembly 1 and a host machine 2. The suction spray assembly 1 includes a housing 11, a suction portion 12 and a spray portion 13. The housing 11 is provided with an installation cavity 11a. The suction portion 12 is connected to the spray portion 13. The suction portion 12 is provided with a suction end exposed in the installation cavity 11a. The spray portion 13 is provided with a spray end exposed in the installation cavity 11a. The host machine 2 includes an air pump 21 and a connection air pipe 22. The connection air pipe 22 connects the air pump 21 and the suction spray assembly 1. The air pump 21 is provided with an air suction port 21a and an air discharge port 21b. The connection air pipe 22 connects the air suction port 21a and the suction portion 12, and the air discharge port 21b communicates the air pump 21 with the outside world.

In an embodiment, the suction spray assembly 1 is composed of a housing 11, a suction portion 12 and a spray portion 13. The housing 11 is made of medical plastic material, such as the acrylonitrile butadiene styrene (ABS) plastic or polypropylene (PP), which has good biocompatibility and chemical resistance, and is easy to process and clean. The suction portion 12 and the spray portion 13 are both partially arranged in the installation cavity 11a of the housing 11, and are respectively provided at the two ends of the installation cavity 11a. The suction portion 12 is provided with a suction end exposed in the installation cavity 11a, and the spray portion 13 is provided with a spray end exposed in the installation cavity 11a. By setting such a layout, the two functional modules can be prevented from interfering with each other during use, thereby improving practicality and efficiency of the device. The user can conveniently use the suction end to clean the nasal cavity or oral cavity, or use the spray end to spray medicine or soften the solidified material in the nasal cavity. Through this design, the spray and suction device for the nasal not only can be used for daily nasal cleaning, but also can be used for medical treatment, such as drug delivery or nasal care.

It should be noted that the detachable spray and suction device for the nasal 100 further includes a host machine 2. The host machine 2 includes an air pump 21 and a connection air pipe 22. The air pump 21 can be a medical air pump 21 with small volume and low noise, which is provided with an air suction port 21a and an air discharge port 21b to ensure the stable supply of airflow. The connection air pipe 22 can be a rubber air pipe or a plastic air pipe with performance of pressure resistance and oil resistance, such as a polyurethane hose or a polyvinyl chloride (PVC) hose. These materials have good sealing performance and chemical resistance, can effectively prevent air leakage, and have a certain oil resistance to cope with the oil that may exist in the air pump 21 or the air tank. The connection air pipe 22 connects the air suction port 21a of the air pump 21 with the suction portion 12 through appropriate joints and clamping devices, to ensure a firm and reliable connection. The air pump 21 is placed at a stable and well-ventilated position. The connection air pipe 22 connects the air suction port 21a of the air pump 21 with the suction portion 12 of the suction spray assembly 1, and the air discharge port 21b directly communicates the air pump 21 with the outside world, to facilitate the discharge of exhaust gas. Through designing the detachable structure, the air pump 21 and the connection air pipe 22 can be separated from the suction spray assembly 1, making the cleaning and disinfection more thorough and avoiding the problem of incomplete cleaning due to the complex structure in the integrated structure. In addition, the connection air pipe 22 is made of materials with performance of pressure resistance and oil resistance, which ensures the stability and safety of air flow transmission and reduces the risk of air leakage. By designing the air suction port 21a and the air discharge port 21b of the air pump 21, air flow supply is stable, which facilitates exhaust gas discharge, thereby improving the practicality and efficiency of the device. In addition, maintenance costs can be reduced since each portion can be replaced separately without replacing the equipment as a whole, thereby extending the service life of the product.

The present application provides a detachable spray and suction device for a nasal 100. By separating the suction spray assembly 1 from the host machine 2, the detachable spray and suction device for the nasal 100 can be used conveniently, and cleaning can be performed more conveniently and thoroughly. The suction spray assembly 1 includes a housing 11, a suction portion 12 and a spray portion 13. The suction portion 12 and the spray portion 13 are respectively provided at the two ends of the housing 11, so that the user can spray and suck the nose according to the needs. The host machine 2 includes an air pump 21 and a connection air pipe 22. The air pump 21 is connected to the suction portion 12 through the connection air pipe 22, to realize the suction function. After use, the user can easily disassemble the suction spray assembly 1 for cleaning without worrying about the damage of non-waterproof components, such as the air pump 21 and the battery, thereby achieving thorough cleaning and disinfection. The detachable design makes it easier to clean the spray and suction device for the nasal 100, avoiding the problem of incomplete cleaning due to the complex structure in the integrated structure. Since the non-waterproof components, such as the air pump 21 and the battery, are separated from the suction spray assembly 1 to be cleaned, the user can fully clean and disinfect the suction spray assembly 1, effectively preventing bacterial growth, reducing hygiene problems, and improving the hygiene of the product and the user experience. In addition, the detachable design may further reduce maintenance costs since each part can be replaced separately without replacing the entire device.

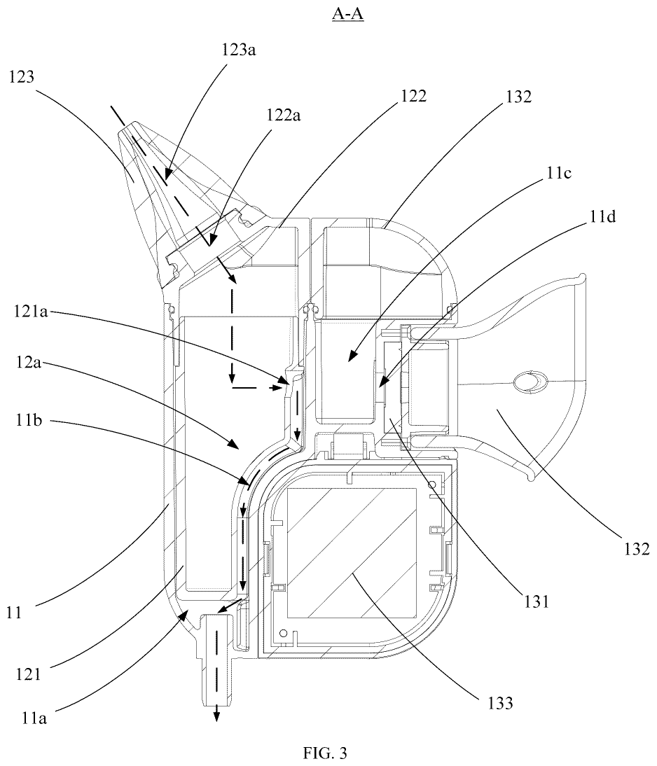

In an embodiment of the present application, as shown in FIG. 3, the suction portion 12 includes a nasal suction chamber 121 and a chamber cover. The chamber cover is connected to the nasal suction chamber 121. The nasal suction chamber 121 is detachably arranged in the installation cavity 11a. The chamber cover and the nasal suction chamber 121 are enclosed to form a buffer cavity 12a, and the chamber cover is provided with a nasal suction port 122a. The nasal suction port 122a communicates the buffer cavity 12a with the outside world.

In an embodiment, the suction portion 12 includes a nasal suction chamber 121 and a chamber cover. The nasal suction chamber 121 is detachably arranged in the installation cavity 11a, so that the user can clean and maintain it after use. The nasal suction chamber 121 and the chamber cover are made of medical plastic materials, such as PP or ABS plastic, which have good biocompatibility and chemical resistance, and are easy to process and clean. The chamber cover is connected to the nasal suction chamber 121 by threads, snaps or bonding, to ensure sealing and stability during use. One end of the nasal suction chamber 121 is provided with a suction end exposed in the installation cavity 11a, which is used to directly contact the user's nasal cavity. The suction end is designed to be rounded to reduce stimulation and damage to the nasal cavity. The other end of the chamber cover is provided with a nasal suction port 122a, and the nasal suction port 122 communicates the buffer cavity 12a with the outside world through a pipe or channel. By setting the buffer cavity 12a, the impact of airflow during direct suction can be reduced, and the use comfort can be improved. The main beneficial effects of this design are that, it provides a suction portion 12 that is easy to disassemble and clean, which not only makes the maintenance of the spray and suction device for the nasal more convenient, but also improves hygiene and safety. By using medical plastic materials, the durability and safety of the device to the human body are ensured. The rounded design of the suction end and the setting of the buffer cavity 12a improve the use comfort and reduce irritation to the nasal cavity, thereby making the device more suitable for sensitive people. In addition, the detachable design further allows the user to replace the suction portion 12 as needed, extending the service life of the product and reducing the cost of long-term maintenance.

In an embodiment of the present application, as shown in FIG. 3, the housing 11 and the nasal suction chamber 121 are enclosed to form an air suction channel 11b. The connection air pipe 22 communicates the air suction channel 11b with the air suction port 21a, and the side wall of the nasal suction chamber 121 is provided with an air suction port 21a, which communicates the buffer cavity 12a with the air suction channel 11b.

In an embodiment, the housing 11 and the nasal suction chamber 121 are enclosed to form an air suction channel 11b. The air suction channel 11b is a key channel connecting the connection air pipe 22 and the air suction port 21a of the air pump 21, and is configured to transmit the airflow from the air pump 21 to the nasal suction chamber 121. The dotted line in the figure shows the air path of the airflow being sucked from the outside world into the nasal suction chamber 121 and the air pump 21. The connection air pipe 22 is the rubber air pipe or plastic air pipe with performance of pressure resistance and oil resistance, such as a polyurethane hose or a PVC hose. These materials not only have good sealing performance and chemical resistance, but also can effectively prevent gas leakage. An air suction port 21a is provided at the side wall of the nasal suction chamber 121. The air suction port 21a directly communicates the buffer cavity 12a with the air suction channel 11b, to ensure the smooth flow of airflow. For application scenarios, such as when performing nasal cleaning or drug treatment, the user can inhale airflow through the nasal suction port 122a. The airflow passes through the air suction channel 11b and enters the buffer cavity 12a, and then is distributed to the nasal cavity through the buffer cavity 12a, so as to achieve the purpose of sucking nasal mucus or delivering drugs. The main beneficial effects of this design are that, it provides an efficient airflow transmission system with a compact structure, which is easy to operate. By integrating the air suction channel 11b and the buffer cavity 12a at the side wall of the nasal suction chamber 121, not only the structure of the device is simplified, but also the efficiency and stability of airflow transmission are further improved, avoiding that foreign matter sucked from the nasal cavity from enters the air pump 21 through the air suction channel 11b. In addition, this design further helps to reduce airflow loss during transmission, ensuring that the airflow can reach the nasal cavity with sufficient pressure and flow, thereby improving the cleaning or treatment effect. In addition, due to the direct connection between the air suction port 21a and the buffer cavity 12a, the potential risk of airflow reflux or leakage is also reduced, thereby enhancing the safety and reliability of the device.

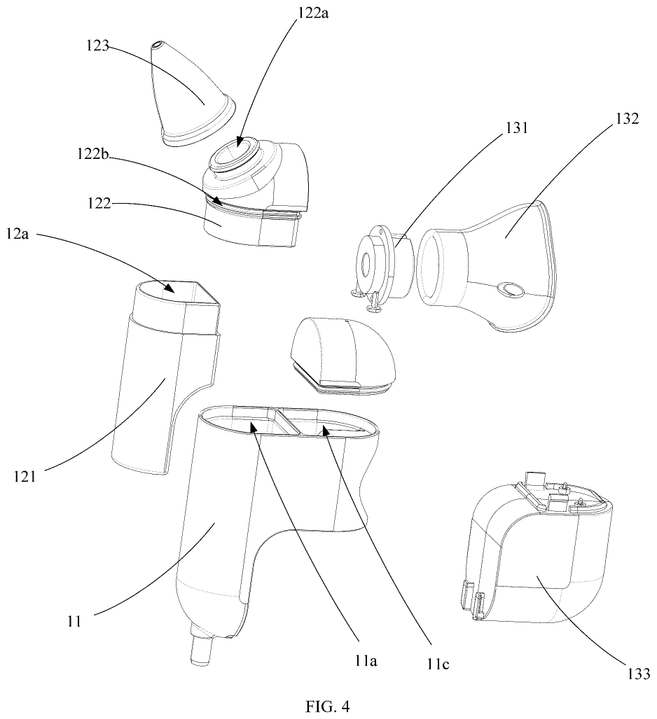

In an embodiment of the present application, as shown in FIG. 3 and FIG. 4, the suction portion 12 further includes a nasal suction tip 123. The nasal suction tip 123 is connected to the chamber cover 122, and a nasal suction channel 123a is provided in the nasal suction tip 123. The nasal suction channel 123a communicates the buffer cavity 12a with the outside world.

In this embodiment, the suction portion 12 includes a nasal suction tip 123 and a chamber cover 122 connected to the nasal suction tip 123. The nasal suction tip 123 is made of medical silicone material, which is soft and human-friendly, and can ensure that the nasal cavity of the user will not be irritated or damaged during use. The nasal suction tip 123 is connected to the chamber cover 122 by threads, buckles or welding, and the like, to ensure sealing and stability during use. The nasal suction tip 123 is provided with a nasal suction channel 123a, which directly communicates the buffer cavity 12a with the outside world. Such a design allows airflow and liquid to flow freely between the buffer cavity 12a and the outside world, thereby realizing the function of sucking nasal mucus. When cleaning the nasal cavity, the user can gently insert the nasal suction tip 123 into the nasal cavity, and use the suction force generated by the air pump 21 to suck the nasal mucus into the buffer cavity 12a through the nasal suction channel 123a. This design makes the process of sucking nasal mucus more efficient and hygienic. By using the nasal suction tip 123 made of medical silicone material, the safety and comfort of the part in contact with the human body are ensured. The close connection between the nasal suction tip 123 and the chamber cover 122 and the direct connection design of the nasal suction channel 123a improve the suction efficiency and reduce the possibility of nasal mucus residue. In addition, this design further helps to reduce the pressure change in the nasal cavity, avoids damage to the nasal mucosa, and improves the user experience and satisfaction.

In an embodiment of the present application, as shown in FIG. 3 and FIG. 4, a liquid storage tank 11c is formed in the installation cavity 11a, and a spray opening 11d is provided at the side wall of the liquid storage tank 11c. The spray portion 13 includes an atomization assembly 131, which is connected to the housing 11, and the atomization assembly 131 is provided at the spray opening 11d.

In an embodiment, the side wall of the liquid storage tank 11c is provided with a spray opening 11d. The core component of the spray portion 13 is the atomization assembly 131, which is connected to the housing 11 and is provided at the spray opening 11d. The atomization assembly 131 can generally be an ultrasonic atomization element or a heating atomization element, which is used to atomize the liquid in the liquid storage tank 11c. In terms of material selection, the liquid storage tank 11c can be made of medical plastics, such as PP or ABS plastics, which have good biocompatibility and chemical resistance, and are easy to process and clean. In the implementation process, the atomization assembly 131 is connected to the housing 11 by threads, snaps, and the like, to ensure sealing and stability during use. The spray opening 11d of the liquid storage tank 11c is designed to match the spray opening 11d, so that the atomized liquid can be sprayed out smoothly. This design improves the atomization efficiency and convenience of use. By accurately providing the atomization assembly 131 at the spray opening 11d, it can be ensured that the atomized liquid is sprayed directly, reducing the residue of the atomized liquid inside the liquid storage tank 11c, and improving the atomization efficiency. In addition, since the connection method between the atomization assembly 131 and the housing 11 is simple and reliable, the user can easily disassemble and clean it, and maintenance is more convenient. In addition, the liquid storage tank 11c made of medical plastic material not only ensures the safety of the product, but also makes the device lighter and more durable.

In an embodiment of the present application, as shown in FIG. 3, the spray portion 13 further includes a spray power structure 133. The spray power structure 133 is detachably connected to the housing 11, and the spray power structure 133 is electrically connected to the atomization assembly 131.

In this embodiment, the spray power structure 133 of the spray portion 13 is detachably connected to the housing 11 to facilitate replacement and maintenance. The spray power structure 133 can be designed as an independent unit, and is electrically connected to the atomization assembly 131 through a standard interface, such as a PH 2.0 3P terminal. This design allows the user to quickly replace the power structure when the power is exhausted without replacing the entire device, which improves the use convenience. The spray power structure 133 is electrically connected to the atomization assembly 131, providing the atomization assembly 131 with the necessary power to achieve atomization of the liquid. The atomization assembly 131 can use a dedicated driver chip for a fixed-frequency atomization sheet, and such a design can ensure a large atomization volume and a stable atomization effect. The atomization assembly 131 can use a microporous atomization sheet, which is formed by bonding a metal microporous sheet and a piezoelectric ceramic sheet. This structure can make the liquid form a mist under the high-frequency resonant oscillation of the piezoelectric ceramic sheet without heating or chemicals. The main beneficial effect of this design is, improving the maintenance convenience and service life of the nasal spray device. The detachable spray power structure 133 allows users to easily replace batteries without professional skills or tools, greatly reducing the difficulty and cost of maintenance. In addition, this design also improves the flexibility of the device, and users can better clean the suction portion 12 and the spray portion 13.

In an embodiment of the present application, as shown in FIG. 3 and FIG. 4, the spray portion 13 further includes an atomization hood 132, which is detachably connected to the housing 11, and is provided at the spray opening 11d.

In an embodiment, the atomization hood 132 is an important component of the spray portion 13, and is detachably connected to the housing 11, which is convenient for users to replace and clean. The atomization hood 132 is usually made of medical materials, such as polyvinyl chloride (PVC), PP, polycarbonate (PC) or silicone rubber, and the like. These materials have good biocompatibility and chemical resistance, and are easy to process and disinfect. The atomization hood 132 is designed to fit tightly to the housing 11, so as to ensure airtightness during spraying. The connection method between the atomization hood 132 and the housing 11 can be threaded connection, snap connection or plug-in connection, and the like. These connection methods are convenient for users to operate and can ensure stability connection. The atomization hood 132 is provided at the spray opening 11d. An atomization assembly 131 is provided between the atomization hood 132 and the liquid storage tank 11c. The atomization assembly 131 is electrically connected to the spray power structure 133 and is configured to convert electrical energy into atomization power. The spray opening 11d of the atomization hood 132 is designed to match the spray direction and flow rate of the atomization assembly 131, so as to ensure the optimization of the atomization effect. In the application scenario, for example, when delivering nasal drugs, the atomization hood 132 can directly deliver the atomized liquid to the user's nasal cavity, thereby improving the drug delivery efficiency and therapeutic effect.

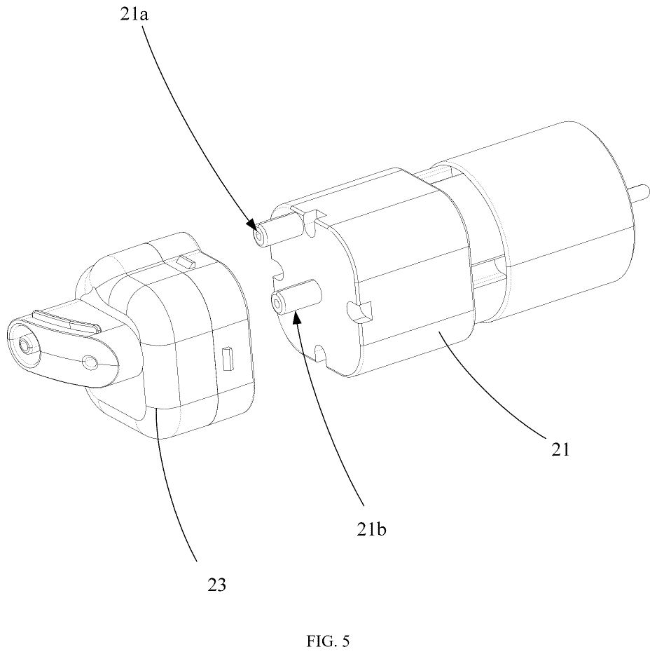

In an embodiment of the present application, as shown in FIG. 5, the host machine 2 further includes an air channel adapter 23, an air channel adapter 23 is provided with an air suction cavity 23a and an air discharge cavity 23b. The air suction cavity 23a communicates the connection air pipe 22 with the air pump 21. The air discharge cavity 23b communicates the air pump 21 with the outside world.

In this embodiment, the air channel adapter 23 of the host machine 2 is a key component, and the air channel adapter 23 is provided with an air suction cavity 23a and an air discharge cavity 23b, which are used to communicate the air pump 21 with the outside world. The air channel adapter 23 is usually made of sound-proof plastic or rubber and other materials, which have good sound insulation and noise reduction functions, and are suitable for the spray and suction device for the nasal. The air suction cavity 23a is configured to communicate the connection air pipe 22 with the air pump 21, and the air discharge cavity 23b is configured to communicate the air pump 21 with the outside world, to ensure smooth airflow. The air channel adapter 23 can be connected to the air pump 21 and the connection air pipe 22 by a face sealing joint or pipe welding, to ensure the stability and sealing of the connection.

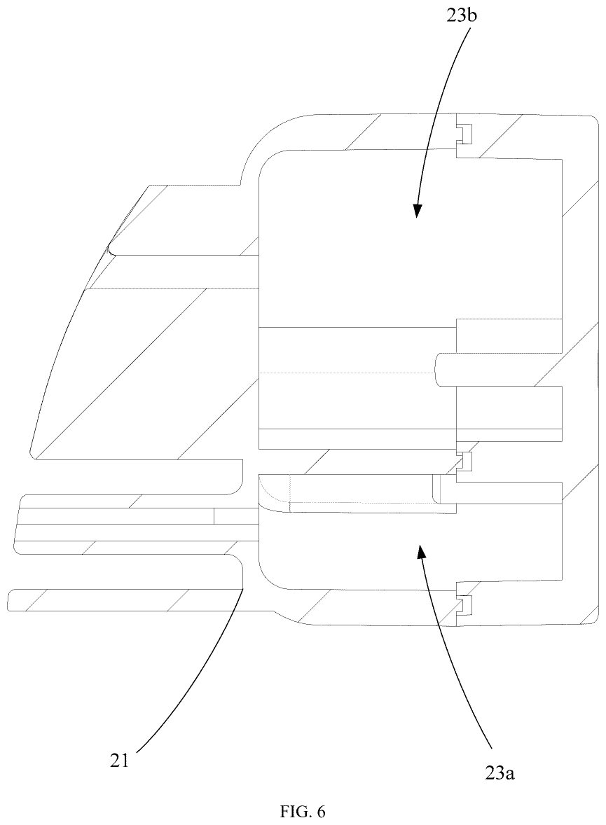

In an embodiment of the present application, as shown in FIG. 5 and FIG. 6, the cross-sectional area of the air suction cavity 23a along the gas flow direction of the air pump 21 is greater than the cross-sectional area of the connection air pipe 22, and the cross-sectional area of the air discharge cavity 23b along the gas flow direction of the air pump 21 is greater than the cross-sectional area of the air discharge port 21b.

In an embodiment, the air channel adapter 23 is designed to optimize the gas flow efficiency and reduce the airflow noise. By setting the cross-sectional area of the air suction cavity 23a being greater than the cross-sectional area of the connection air pipe 22, and setting the cross-sectional area of the air discharge cavity 23b being greater than the cross-sectional area of the air discharge port 21b, the pressure loss and noise of the air flow can be reduced when the air flow passing through these components, and the air suction efficiency and exhaust efficiency of the air pump 21 can be improved. The air channel adapter 23 can be made of medical materials such as ABS plastic or PP, which have good mechanical properties and chemical resistance and are suitable for medical equipment. In the implementation process, the air suction cavity 23a is connected to the air suction port 21a of the air pump 21 through the connection air pipe 22, and the air discharge cavity 23b is connected to the air discharge port 21b of the air pump 21 through the discharge pipe, so as to achieve smooth flow of air. This design allows the exhaust gas generated by the air pump 21 during operation to be discharged directly without accumulating inside the device, ensuring the unidirectional flow of the air flow and the hygiene of the device. The main beneficial effects of this design are, improving airflow efficiency and reducing the energy consumption of the air pump 21. By increasing the cross-sectional areas of the air suction cavity 23a and the air discharge cavity 23b, the resistance of the airflow during passing the cavities can be reduced, thereby reducing the energy consumption and noise of the air pump 21. In addition, this design also helps to improve the air suction efficiency and exhaust efficiency of the air pump 21, so that the nasal spray device can work more efficiently. In addition, a reasonable airflow channel also helps to reduce the wear of the air pump 21 and extend the service life of the device. By optimizing the design of the air channel adapter 23, the performance of the entire nasal spray device can be improved, and a more comfortable and quiet use experience can be provided to the user.

In an embodiment of the present application, as shown in FIG. 5, the air channel adapter 23 is made of ethylene-vinyl acetate copolymer (EVA) or silicone.

In this embodiment, the air channel adapter 23 is a key component in the spray and suction device for the nasal, and its material is EVA or silicone. Both materials have good noise reduction performance, flexibility and chemical resistance, and are suitable for air path connection of the medical equipment. In the implementation process, the air channel adapter 23 may be a structure including an air suction cavity 23a and an air discharge cavity 23b. The cross-sectional area of the air suction cavity 23a is greater than the cross-sectional area of the connection air pipe 22, and the cross-sectional area of the air discharge cavity 23b is greater than the cross-sectional area of the air discharge port 21b. In this way, the pressure loss and noise of the air flow when passing through these components can be reduced, and the suction efficiency and exhaust efficiency of the air pump 21 can be improved. The air channel adapter 23 can be made into the desired shape by injection molding. The air channel adapter 23 made of EVA can be connected by the aseptic connection method of the EVA pipeline, while the air channel adapter 23 made of silicone can be formed into an elastic soft structure by vulcanization process in view of utilizing the high temperature resistance. The air channel adapter 23 made of EVA or silicone can ensure the flexibility and durability of the air channel adapter 23, and is also easy to disinfect and clean, thereby improving the hygienic and safety of medical equipment. The low melting point of EVA material enables it to fuse quickly when connecting the pipe, thereby improving the efficiency of connecting the pipe. The high temperature resistance, environmental protection, non-toxic and odorless features of silicone enable it to maintain stable performance during high temperature disinfection, as well as being safe and harmless to the human body. In addition, the air channel adapter 23 made of these two materials can effectively reduce airflow noise and improve the working efficiency of the air pump 21, thereby providing users with a more comfortable and quiet use experience.

The above are only some embodiments of the present application, and do not limit the scope of the present application thereto. Under the concept of the present application, any equivalent structural transformation made according to the description and drawings of the present application, or direct/indirect application in other related technical fields, shall fall within the scope of the present application.

What is claimed is:

1. A detachable spray and suction device for a nasal, comprising:
a suction spray assembly comprising a housing, a suction portion, and a spray portion, wherein the housing is provided with an installation cavity, and the suction portion is connected to the spray portion; the suction portion is provided with a suction end exposed in the installation cavity, and the spray portion is provided with a spray end exposed in the installation cavity; and
a host machine comprising an air pump and a connection air pipe, wherein the connection air pipe is connected with the air pump and the suction spray assembly, and the air pump is provided with an air suction port and an air discharge port; the connection air pipe is connected with the air suction port and the suction portion, and the air discharge port is configured to communicate the air pump with outside world.

2. The detachable spray and suction device for the nasal according to claim 1, wherein the suction portion comprises a nasal suction chamber and a chamber cover; the chamber cover is connected to the nasal suction chamber, and the nasal suction chamber is detachably provided in the installation cavity; the chamber cover and the nasal suction chamber are enclosed to form a buffer cavity; the chamber cover is provided with a nasal suction port, and the nasal suction port is configured to communicate the buffer cavity with the outside world.

3. The detachable spray and suction device for the nasal according to claim 2, wherein the housing and the nasal suction chamber are enclosed to form an air suction channel, and the connection air pipe is configured to communicate the air suction channel with the air suction port; a side wall of the nasal suction chamber is provided with an air suction port, and the air suction port configured to communicate the buffer cavity with the air suction channel.

4. The detachable spray and suction device for the nasal according to claim 3, wherein the suction portion further comprises a nasal suction tip, and the nasal suction tip is connected to the chamber cover; the nasal suction tip is provided with a nasal suction channel, and the nasal suction channel is configured to communicate the buffer cavity with the outside world.

5. The detachable spray and suction device for the nasal according to claim 1, wherein a liquid storage tank is provided in the installation cavity; a spray opening is provided at a side wall of the liquid storage tank, and the spray portion comprises an atomization assembly; the atomization assembly is connected to the housing, and the atomization assembly is provided at the spray opening.

6. The detachable spray and suction device for the nasal according to claim 5, wherein the spray portion further comprises a spray power structure; the spray power structure is detachably connected to the housing, and the spray power structure is electrically connected to the atomization assembly.

7. The detachable spray and suction device for the nasal according to claim 6, wherein the spray portion further comprises an atomization hood; the atomization hood is detachably connected to the housing, and the atomization hood is provided at the spray opening.

8. The detachable spray and suction device for the nasal according to claim 1, wherein the host machine further comprises an air channel adapter, and the air channel adapter is provided with an air suction cavity and an air discharge cavity; the air suction cavity is configured to communicate the connection air pipe with the air pump, and the air discharge cavity is configured to communicate the air pump with the outside world.

9. The detachable spray and suction device for the nasal according to claim 8, wherein a cross-sectional area of the air suction cavity along an air flow direction of the air pump is larger than a cross-sectional area of the connection air pipe, and a cross-sectional area of the discharge cavity along the air flow direction of the air pump is larger than a cross-sectional area of the discharge port.

10. The detachable spray and suction device for the nasal according to claim 9, wherein the air path adapter is made of an ethylene-vinyl acetate copolymer (EVA) material or a silicone material.
